(19) United States
(12) Patent Application Publication
(10) Pub. No.: US 2024/0060026 A1
(43) Pub. Date: Feb. 22, 2024

(54) MANUFACTURABLE CO-CULTURE MODULE

(71) Applicant: Cerillo, Inc., Charlottesville, VA (US)

(72) Inventors: Kevin Seitter, Charlottesville, VA (US); Andrew Homyk, Charlottesville, VA (US); Thomas Moutinho, JR., Charlottesville, VA (US); Kristin Schmidt, Charlottesville, VA (US); Sruthi Gopinathan, Charlottesville, VA (US); John Peck, Charlottesville, VA (US)

(21) Appl. No.: 18/452,076

(22) Filed: Aug. 18, 2023

Related U.S. Application Data
(60) Provisional application No. 63/371,763, filed on Aug. 18, 2022.

(51) Int. Cl. C12M 3/00; C12M 1/00; C12M 1/32

(52) U.S. Cl. CPC C12M 23/44; C12M 23/22; C12M 29/04; C12M 23/12

(57) ABSTRACT

Herein are described co-culture modules having a plurality of sample volumes separated by vertical semi-permeable membranes. The co-culture modules are typically manufacturable and can be single use or reusable.

### CROSS REFERENCE TO RELATED APPLICATIONS

This application is entitled to priority pursuant to 35 U.S.C. § 119(e) to U.S. provisional patent application No. 63/371,763, filed on 18 Aug. 2022. The contents of which are incorporated herein by reference in their entirety.

### BACKGROUND OF THE INVENTION

In a co-culture, interactions between a plurality of species are analyzed. Generally, microbes grown in a co-culture can be either mixed within the same growth media, grown within the same media but separated by a media exchange membrane, or grown separately with exposure to products generated by the other microbe(s). The studies of such cultures go beyond cultures grown in isolation in that they more accurately represent real world phenomena. The analysis of co-cultures provides a better understanding of microbial dynamics in emerging fields of study, such as the gut microbiome. The advancement of co-culture laboratory techniques improves the ability to fight infection, treat disease, and proactively manage holistic human health.

Currently, there is no recognized industry standard for co-culture evaluation in a form factor that allows for real-time quantitative assessment of the phenotypic behavior of the individual strains in co-culture. Most simply, co-culture can be studied in a mixed condition. In this case, both cultures share the same space and the same media. This experiment, however, does not allow the measurement of individual cultures' growth.

Plate colony assays may also be used as a co-culture technique. This technique involves culturing on a dish and observing the interactions between visible colonies of microbes. This is, however, not quantitative and is also limited in its application to real-world microbial environments. It also does not apply to cultures grown in suspension in liquid media.

In another co-culture technique, vertically stacked cultures can be grown in an arrangement in which a horizontal membrane separates the cultures but allows for media exchange. The horizontal membrane and upper chamber can be removed, creating a way to individually read optical density for growth measurement experiments. However, this poses a contamination risk during manipulation, and does not effectively separate two suspension cultures; the membrane insert is designed for adherent cells, to measure how they interact with a suspension culture in the lower chamber. As such, the upper culture is generally not a microbial culture, but some sort of mammalian or other eukaryotic sample. These inserts are also restricted in their capacity to adequately characterize phenotypic growth dynamics, and such measurement cannot be done in real-time due to the vertically stacked geometry. However, this is currently the most common form of co-culture study, and the most well-known brand is the Corning TransWell system.

A final example of a co-culture technique is a serial media exchange protocol, or so-called “spent media” experiment. In this method, a microbial culture is grown, and its media after growth is sterilized and re-used to grow a second microbial culture. In this way, the metabolic products of the first microbe can be observed to influence the behavior of the second microbe. This technique allows quantification of the phenotypic behavior of each culture, but it cannot capture real-time dynamics. The single timepoint of exchange is also highly dissimilar to the continuous exchange that takes place in the real world (and even in other co-culture techniques).

Specialty co-culturing systems available to microbiologists and pathologists typically exhibit features that block the ability to make optical reads on the plates in real-time. It is desirable for the researcher to be able to monitor their cultures dynamically, in a repeatable manner resembling a multiwell plate in a plate reader while simultaneously being able to determine contact-independent interactions. As the field of co-culture microbiology continues to expand in its breadth and application it is imperative that a standard system for performing these measurements be established, so that scientists can compare and contrast their findings. The invention presented herein intends to become that standard for dynamic and repeatable co-culture measurement.

### SUMMARY OF THE INVENTION

In an aspect, there is described a co-culture module, comprising a central volume, end cap volumes, and semi-permeable membranes, configured such that the module can interface with a multiwell plate reader.

In another aspect, there is described a co-culture module that is manufacturable.

In another aspect, there is described a co-culture module, comprising a plurality of bodies forming a plurality of sample wells, at least one alignment feature between the bodies, and at least one semi-permeable membrane affixed between the bodies.

In another aspect, there is described a co-culture module, comprising a pair of bodies forming two sample wells, with at least one alignment feature between the bodies, and one semi-permeable membrane affixed between the bodies.

These and other aspects, which will become apparent in the following detailed description, have been achieved by the inventors' discovery of a novel co-culture module.

### BRIEF DESCRIPTION OF THE DRAWINGS

FIG. 1. is an isometric view of an assembled co-culture module 100 depicting a central volume 400, two end cap volumes 200, and two semi-permeable membranes 300, such module being configured to interface with a multiwell plate reader.

FIG. 2. is an isometric, exploded view of a co-culture module configured to interface with a multiwell plate reader.

FIG. 3. is an isometric view of the under-side of an assembled co-culture module showing an internal rib network 210 and flat regions for sample interrogation (e.g., 212).

FIG. 4A. is an isometric section view of a co-culture module with a section expanded to a detailed view (right) that highlights the manufacturing features used to secure and hermetically seal the module.

FIG. 4B. is an isometric section view of a co-culture module which provides a pair of sample volumes, that highlights the manufacturing features used to secure and hermetically seal the module.

FIG. 5. is a top down view of an end cap 200 illustrating well coordinate labels 220.

FIG. 6. is an isometric view of a co-culture module which provides a pair of sample volumes.

FIG. 7. is an isometric, exploded view of a co-culture module which provides a pair of sample volumes, highlighting manufacturing features used to secure and hermetically seal the module.

FIG. 8. is an isometric view of the under-side of an assembled co-culture module which provides a pair of sample volumes.

### DETAILED DESCRIPTION OF THE INVENTION

Exemplary aspects of the present invention are described herein. Although the following detailed description contains many specifics for purposes of illustration, a person of ordinary skill in the art will appreciate that variations and alterations to the following details are within the scope of the invention. Accordingly, the following aspects of the invention are set forth without any loss of generality to, and without imposing limitations upon, the claimed invention.

An aspect involves an apparatus with a vertically oriented semi-permeable membrane positioned between one or more pairs of wells on a single module. These vertically oriented membranes allow for the selective transfer of molecules and/or microbes dependent upon the porosity, thickness, and material selected for the membrane.

Another aspect involves a multi-cavity co-culture module, comprising:
a) a plurality of bodies, each containing at least one sample well, wherein the bodies are precisely aligned with each other, such that a plurality of sample wells in said bodies are connected via horizontal cavities;
b) at least one alignment feature located between the bodies to facilitate the bodies' alignment and hermetic sealing by way of ultrasonic welding, heat stamping, laser welding, press fitting, or other industrial process for permanent adhering; and,
c) at least one semi-permeable membrane permanently sealed to or between the bodies, aligned so as to completely cover at least one horizontal cavity connecting said sample wells;
wherein:
the membrane material is of a porosity that selectively enables the transfer of biologically relevant compounds between wells while preventing the transfer of organisms between wells; and,
the bodies and membranes are permanently affixed to one another prior to use to form a single operable piece.

In another aspect, the sample wells are designed to present at horizontal locations corresponding to the horizontal locations of one or more wells of a standard multiwell plate.

In another aspect, a mechanical feature is presented to facilitate the alignment and temporary attachment of a horizontal cover over the top of the module. Examples of the horizontal cover include a rigid lid and a membrane.

In another aspect, the horizontal surfaces are optically clear to facilitate the measurement of optical density, absorbance, transmittance, and/or other properties involving the transmission of light through the sample.

In another aspect, the vertical surfaces are optically opaque to facilitate the measurement of fluorescence, luminescence, and/or other properties involving the emission of light from the sample.

In another aspect, the industrial process used to fix the bodies together accomplishes the hermetic sealing with the membrane material in the absence of other elements (e.g., such as a gasket).

In another aspect, the industrial process used to fix the bodies together is ultrasonic welding.

In another aspect, the membrane is permanently affixed to at least one body via heat sealing prior to the bodies being permanently affixed to one another.

In another aspect, the bodies are subsequently affixed to one another using ultrasonic welding.

Affixing a membrane vertically, in a manufacturable way, presents a unique challenge to one skilled in the art. Not only does the sometimes thin and fragile membrane need to be precisely positioned, but it also requires a hermetic seal between itself and multiple mating components without damaging the membrane or changing its permeability features. Thus, another aspect involves the manufacturing processes and component features that can be employed to produce co-culturing systems with a vertically oriented membrane.

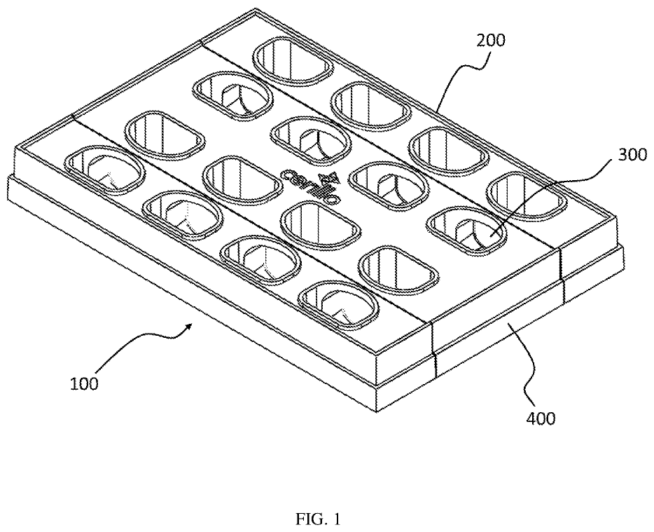

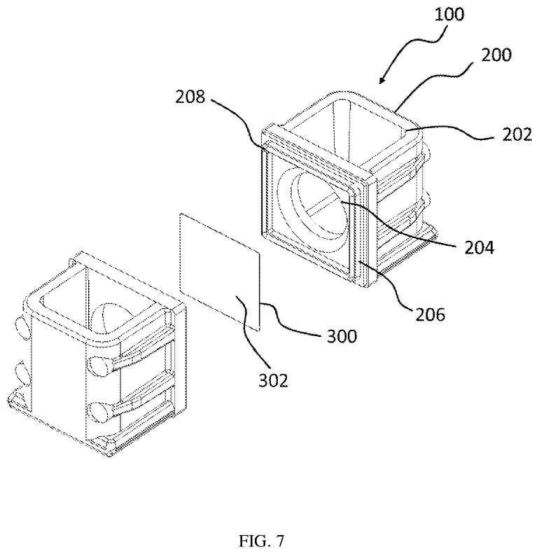

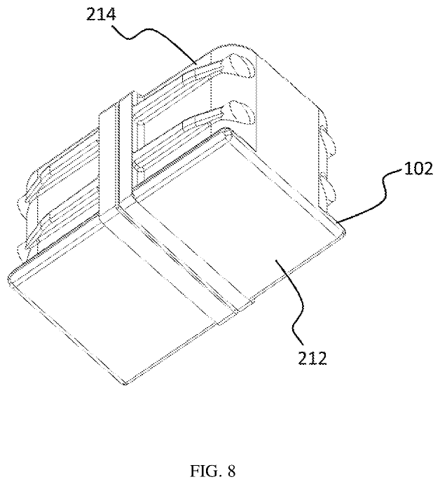

Names for components shown in the FIGS. 1-8 are as follows:

| | |
|---|---|
| 100 | Disposable Co-Culture Module |
| 200 | End Cap Volume |
| 300 | Semi-Permeable Membrane |
| 400 | Central Volume |
| 102 | Module Lower Perimeter Embossment |
| 104 | Module Perimeter Ridge |
| 106 | Module Sealing Interface |
| 202 | End Cap Well |
| 204 | End Cap Well Membrane Cutout |
| 206 | Volume Alignment Feature |
| 208 | Welding Protuberance |
| 210 | Internal Rib Network |
| 212 | Exterior Transduction Modality Window |
| 214 | Module Perimeter Recess |
| 218 | Interior Transduction Modality Window On End Cap Volume |
| 220 | Well Coordinate Labels |
| 222 | Individual Well Ridge |
| 302 | Membrane Form |
| 304 | Membrane Alignment/Securement Holes |
| 306 | Membrane Installed Into Assembled Co-Culture Module |
| 402 | Membrane Fixation Protuberance/Recess |
| 404 | Central Well Membrane Cutout |
| 406 | Volume Alignment Holes With Material Trap |
| 408 | Central Well |
| 410 | Material Shell-Out |
| 412 | Interior Transduction Modality Window On Central Volume |
| 414 | Spill Trap Recess And Logo Placement Region |

As depicted in FIGS. 1-8, another aspect involves a module, comprising: a disposable co-culture module 100, one or more end cap volumes 200, one or more semi-permeable membranes 300, and up to one or more central volumes 400. In an aspect, the co-culture module 100 is assembled in such a way as to form a contiguous body with an exterior perimeter 102 that is configured to fit as assembled with (not shown) or without a lid into instruments and devices common to laboratory and diagnostic practice for measuring microplates and analogous products.

Orientation and manipulation of commonly thin and fragile semi-permeable membranes 300 presents a unique assembly challenge, especially in a vertical or near vertical orientation designed to be secured between two or more rigid bodies. A benefit of the co-culture module described herein arises from the types of features, materials, and configuration of components used to make the co-culture module assembly 100 manufacturable at scale.

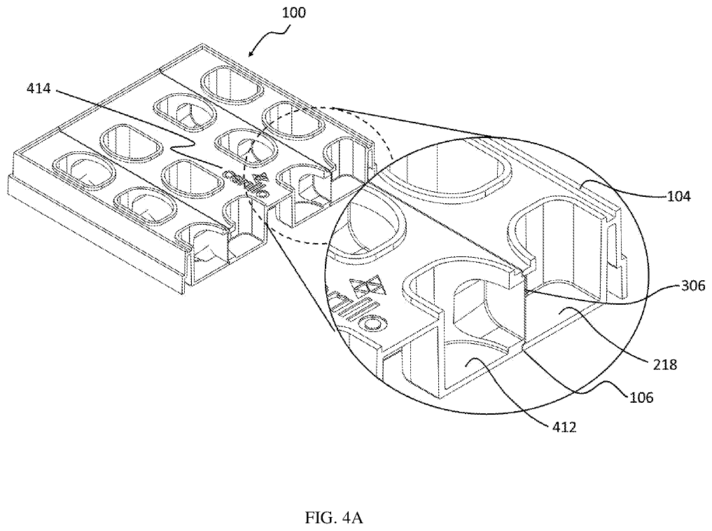

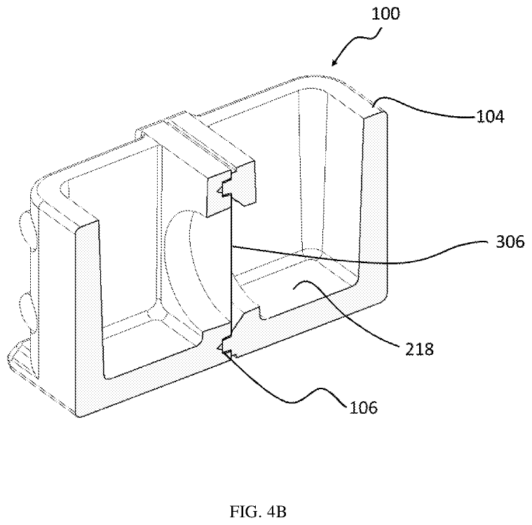

Another aspect involves a disposable and manufacturable co-culture module 100, designed to interface with a multiwell plate reader, the module, comprising: one or more of the following:
a) at least one contiguous feature defining the bottom exterior perimeter of the co-culture module 102;
b) at least one feature defining the top exterior perimeter of the co-culture module 104;
c) at least one feature configured to bond, hermetically seal, or affix different module components together 106;
d) at least one end cap volume 200, comprising one or more of the following:
   i) at least one sample well 202 for holding a microbial sample; and,
   ii) at least one cavity 204 in a side facing wall configured to interface with the semi-permeable membrane 300;
   iii) at least one feature for alignment 206 with other components in the co-culture assembly 100;
   iv) at least one feature configured for plastic welding or affixation 208 of the end cap volume 200 with other components in the co-culture assembly 100 using a manufacturing method; and,
   v) at least one window or other feature 218 to allow for the transduction of sample parameters within the sample well 202;
e) at least one semi-permeable membrane 300, comprising: one or more of the following:
   i) a membrane form 302, configured to be aligned and vertically or near-vertically oriented between volumes 306 in the co-culture module (see FIGS. 4A and 4B); and,
   ii) at least one feature 304 configured to align the semi-permeable membrane form 302 with alignment features on either an end cap volume 200, and/or a central volume 400;
f) at least one central volume 400, comprising: one or more of the following:
   i) at least one feature configured for plastic welding or affixation 402 of the central volume/s 400 to other components in the co-culture assembly 100;
   ii) at least one cavity 404 in a side facing wall configured to interface with the semi-permeable membrane 300;
   iii) at least one feature for alignment 406 with other components in the co-culture assembly 100;
   iv) at least one sample well 408 for holding a microbial sample;
   v) at least one window or other feature 412 to allow for the transduction of sample parameters within the sample well 408;
   vi) an area designated for the placement of a company logo or an inscribable plaque for writing or printing experimental information 414; and,
   vii) a region of recess 414 to capture a spilled sample or serve as a basis of mechanical attachment for a lidding component or sample cover.

In another aspect, the lidding component or sample cover is a horizontal cover.

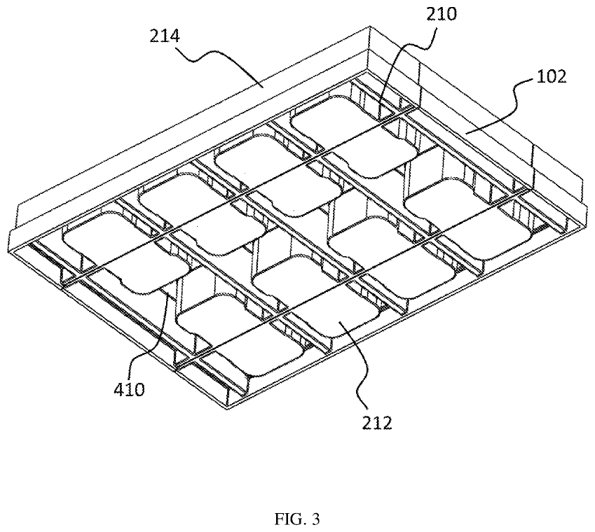

Referring to FIG. 3, in another aspect, the co-culture module using an exterior rim 102 is configured with geometries intended to fit a variety of laboratory plate readers or instruments. Additionally, the exterior geometry can be other than rectangular to function and register correctly within plate reader instruments (not shown).

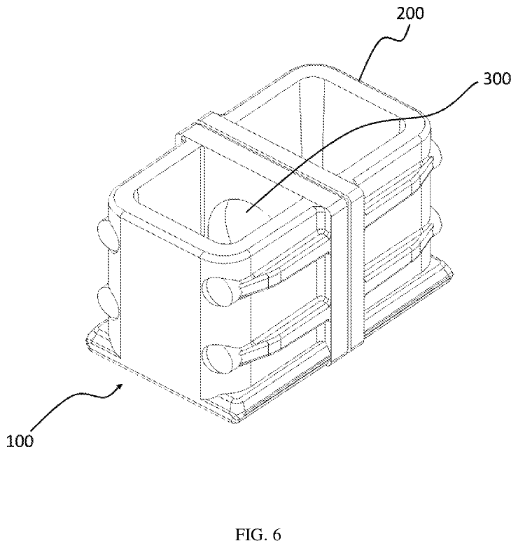

Another aspect involves a co-culture module composed of different combinations and permutations of the assembly described herein. An example is a co-culture module comprising two end caps 200 with no central volume 400 (FIG. 6). Another example is a co-culture module configured to be constructed using three or more end caps arrayed in a geometric pattern around at least one central volume 400 (not shown).

Another aspect involves co-culture modules that are not restricted to pairs of individual wells in fluidic communication with a semi-permeable membrane (not shown). An example is a co-culture module comprising three or more wells connected in series to each other, forming a network of samples connected by semi-permeable membranes. Another example is a co-culture module wherein more than one well is connected to a single communal well by semi-permeable membranes.

In an aspect where a lid (e.g., a rigid lid) is used with module 100, the module comprises a contiguous exterior ridge 104 (see FIGS. 4A and 4B) designed to elevate and constrain the lid. Another aspect involves the ridge acting to contain spills on the top surface of the assembled co-culture module 100.

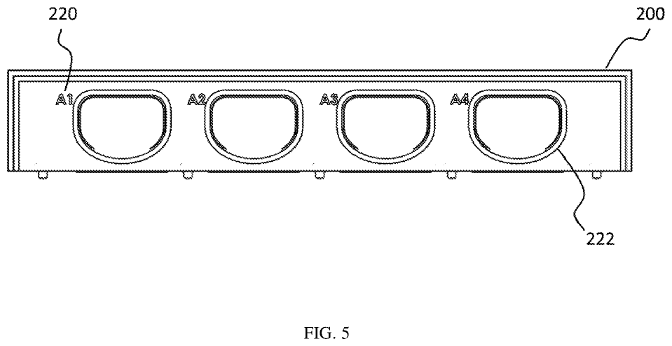

Another aspect involves a co-culture module where the wells on each volume are labeled with alpha-numeric indicators of the well positions within the assembly (see FIG. 5, 220). Another aspect involves these indicators being recessed or raised portions of the volume, screen printed, or, alternatively, left as a blank textured surface where the user could apply their own label.

Another aspect involves wells of either the central 400 or end cap volumes 200 having raised regions (see FIG. 5, 222) configured to align and contact a lid, or act as a barrier to reduce the effect of evaporation of a contained liquid sample. Another aspect involves these raised surfaces 222 acting in conjunction with a raised exterior perimeter 104 (see FIG. 4A, 104) to contain spills or prevent sample contamination from one well to another.

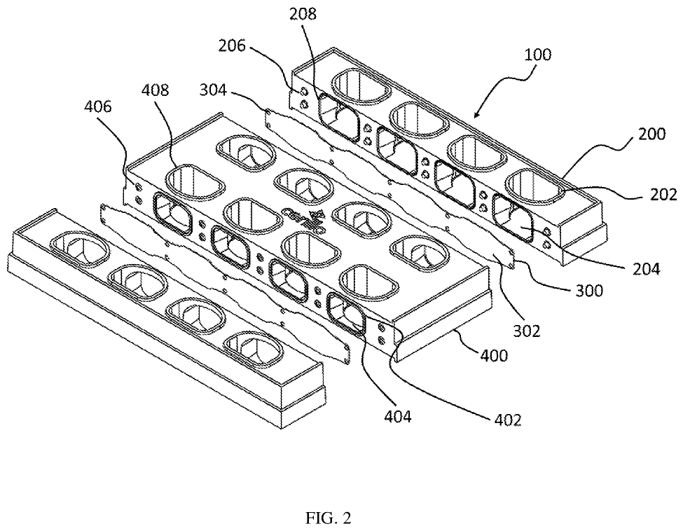

One of the most challenging aspects of the co-culture modules described herein is the combination of manufacturing practices that must be used to cut, seal, position, and align or affix the semi-permeable membrane 300 to the various volumes in the co-culture module assembly. FIGS. 2 and 4A demonstrate an aspect of a manufacturing solution where a membrane body 306 is affixed to a central volume 400 using a heat sealing process to affix it to a flat surface 402 encircling a central well membrane cutout 404. The heat sealing process forms a hermetic seal between the membrane and the central volume sealing those two components together. Once the central volume and membrane are hermetically sealed, the end cap 200 is then affixed to the assembly with the use of an assembly feature 208. FIG. 4B demonstrates a similar aspect of a manufacturing solution of a module consisting only of a pair of wells.

Further aspects involve assembly methods to seal an end cap to the central volume membrane assembly that include one or more of the following.
a) A ridge feature on the end cap 208 that is compressed onto another adjoining feature 402 on the central volume using a static load. These features, in an aspect, are then plastically welded together using ultrasonic welding or a similar vibratory process to form a hermetic seal between the end cap 200 and the central volume 400. In such an aspect, flat surfaces can be created and supported with ribs on the exterior surface of the end cap 214 (see FIG. 3) to support assembly using an ultrasonic horn or other vibratory implement.
b) A gasket groove formed around the central well membrane cutout 404 or in a similar location around the periphery of the end cap well membrane cutout 204 (see FIG. 2). In this aspect, an O-ring or similar gasket material is positioned into the gasket groove and components ultrasonic welded together with protuberances 206 and mating features on the opposite component meant to interface with the protuberances during the welding process 406. Likewise, instead of using an ultrasonic welding process a similar method can be used to mechanically affix components together such as heat staking or other similar technology used to form a mechanical bond between rigid plastic components.
c) Two flat surfaces in the positions of 208 and its mating element on the central volume 400 are sealed together using a laser welding process.
d) An adhesive or gasket is affixed to the membrane form 302. This adhesive or gasket forms a hermetic seal on one or more faces when assembled between a combination of end caps 200 and/or central volumes 400.

Another aspect involves the end cap 200 and central volume 400 configured as a single component (not shown). In this aspect, the membrane form 302 is rigidly affixed to a frame designed to be assembled between adjoining well pairs or other combinations of wells.

Another aspect involves end caps 200 being hermetically sealed to the central volume/s 400 using a plastic assembly method such as laser welding, ultrasonic welding, or other practice described herein. In this aspect, a slot or recess is configured between the aforementioned components to house either an individual membrane 302 or a combination of membrane assemblies, either as a raw material or affixed to a rigid frame. Another aspect involves these membranes being either permanently affixed to the co-culture assembly or configured in a way that they are removable so that the module could be sterilized and reused. Alternatively, in another aspect, the modules are configured for single use.

Another aspect involves the individual components making up the assembly of the module such as the end caps 200 or the central volumes 400 configured so that they are constructed from different materials or combinations of treatments to allow for various types of experiments to be run with a single module. One example is an end cap with opaque walls—designed for a fluorescence assay—in combination with a central volume with clear, tissue-culture-treated walls.

Another aspect involves the end caps 200 and/or the central volumes 400 being injection molded.

Another aspect involves the membrane form 302 being molded in place onto either the end cap 200 or the central volume 400 or both. In the case where the membrane form is molded into both, the end cap and central volume would be configured to be a single component.

All references listed herein are individually incorporated herein in their entirety by reference. Numerous modifications and variations of the present invention are possible considering the above teachings. It is therefore to be understood that within the scope of the appended claims, the invention may be practiced otherwise than as specifically described herein.

1. A multi-cavity co-culture module, comprising:
a) a plurality of bodies, each containing at least one sample well, wherein the bodies are precisely aligned with each other, such that a plurality of sample wells in said bodies are connected via horizontal cavities;
b) at least one alignment feature located between the bodies to facilitate the bodies' alignment and hermetic sealing by way of ultrasonic welding, heat stamping, laser welding, press fitting, or other industrial process for permanent adhering; and,
c) at least one semi-permeable membrane permanently sealed to or between the bodies, aligned so as to completely cover at least one horizontal cavity connecting said sample wells;
wherein:
the membrane material is of a porosity that selectively enables the transfer of biologically relevant compounds between wells while preventing the transfer of organisms between wells; and,
the bodies and membranes are permanently affixed to one another prior to use to form a single operable piece.

2. The module of claim 1, wherein the module is configured for single use.

3. The module of claim 1, wherein the module is configured to be sterilizable and reusable.

4. The module of claim 1, wherein the sample wells are designed to present at horizontal locations corresponding to the horizontal locations of one or more wells of a standard multiwell plate.

5. The module of claim 4, wherein the external dimensions of the module match those of a standard multiwell plate.

6. The module of claim 1, wherein the sample wells are arranged in pairs.

7. The module of claim 1, wherein at least one sample well is configured to be in fluidic communication with at least two other sample wells to form a co-culturing network of more than two wells.

8. The module of claim 1, wherein a mechanical feature is presented to facilitate the alignment and temporary attachment of a horizontal cover over the top of the module.

9. The module of claim 8, wherein the horizontal cover is a rigid lid.

10. The module of claim 8, wherein the horizontal cover is a membrane.

11. The module of claim 1, wherein the horizontal surfaces are optically clear to facilitate the measurement of optical density, absorbance, transmittance, and/or other properties involving the transmission of light through the sample.

12. The module of claim 1, wherein the vertical surfaces are optically opaque to facilitate the measurement of fluorescence, luminescence, and/or other properties involving the emission of light from the sample.

13. The module of claim 1, wherein a gasket is used to form the hermetic seal between bodies and membrane and is permanently held in place by the seal between the bodies.

14. The module of claim 1, wherein the industrial process used to fix the bodies together accomplishes the hermetic sealing with the membrane material in the absence of other elements.

15. The module of claim 14, wherein the industrial process used to fix the bodies together is ultrasonic welding.

16. The module of claim 1, wherein the membrane is permanently affixed to at least one body via heat sealing prior to the bodies being permanently affixed to one another.

17. The module of claim 16, wherein the bodies are subsequently affixed to one another using ultrasonic welding.
